(19) United States
(12) Patent Application Publication
(10) Pub. No.: US 2023/0146816 A1
(43) Pub. Date: May 11, 2023

(54) LARGE AREA SURFACE SAMPLER WITH INVERTIBLE BAG

(71) Applicant: INNOVAPREP LLC, Drexel, MO (US)

(72) Inventors: Michael Fischer, Lee's Summit, MO (US); Bryan Long, Odessa, MO (US); Steven D. Graham, Drexel, MO (US); Sidney J. Spry, Lexington Park, MD (US); Alec Adolphson, Raymore, MO (US); John D. Birkenholz, Kansas City, MO (US); Andrew E. Page, Smithton, MO (US); David S. Alburty, Drexel, MO (US)

(21) Appl. No.: 17/984,204

(22) Filed: Nov. 9, 2022

Related U.S. Application Data
(60) Provisional application No. 63/277,573, filed on Nov. 9, 2021.

(51) Int. Cl. B01L 3/00

(52) U.S. Cl. CPC B01L 3/505

(57) ABSTRACT

Devices, systems, and methods are disclosed which relate to using a use large area surface sampler with invertible bag for collection of biological and non-biological particles prior to subsequent analysis. The large area surface sampler with invertible bag uses an inverted bag with bonded sampling material to first serve as a sampling device followed by the bag being turned right side out and the captured particles being extracted inside of the bag. The device includes measures for sealing liquid in the sampling material using a protective cup at time of manufacture. This enables users to simply remove the protective cup and perform wet surface sampling prior to turning the bag right side out and removing captured particles from the sampling material with repeated compressions. The described device, systems and methods allow users to capture and elute surface samples for human clinical, veterinary, food safety, pharmaceutical, outbreak investigations, forensics, biodefense and bioterrorism response, environmental monitoring, and other applications where collection of samples from surfaces and humans or animals is required.

This Patent application claims priority to U.S. Provisional Patent Application Ser. No. 63/277,573, filed Nov. 9, 2021, the content of which is hereby incorporated by reference herein in its entirety into this disclosure.

### GOVERNMENT INTERESTS

This subject disclosure was funded in-part under a DOD contract, under contract number W911SR-17-C-0056. The government may have certain rights in this subject disclosure.

### BACKGROUND OF THE SUBJECT DISCLOSURE

### Field of the Subject Disclosure

The subject disclosure relates generally to the collection of samples from surfaces. More particularly, the subject disclosure relates to devices, systems, and methods for sampling of large surface areas for biological particles and extraction of the biological particles into a small liquid volume for subsequent analysis.

### Background of the Subject Disclosure

A wide range of existing, and developing, rapid analysis platforms are potentially useful technologies for detection and identification needs. Detection and identification may key on whole organisms, nucleic acids, or proteins. Culture based analysis, antibiotic susceptibility testing, and functional assays all require live organism samples. Common nucleic acid techniques include qPCR, UHTS, and hybridization arrays. ELISA and other immunoassay techniques, mass spectrometry, chromatography techniques, and other techniques may be used for protein analysis. There are significant reasons in some cases to choose one of these techniques over the other or in some cases to analyze with more than one technique. The systems and methods are powerful techniques for rapidly detecting and identifying pathogens and other microorganisms and biological particles, but they require good techniques and devices for efficiently delivering high-quality samples, contained target organism in a small sample volume.

Swabs and wipes are routinely used as tools for collection of biological and non-biological materials from environmental and clinical samples. Samples are collected by swabbing or wiping an appropriate surface. The sample must then be recovered from the collection tool into a liquid buffer or in some cases directly onto a culture plate for biological samples. A number of conventional methods exist for recovery of samples from swabs and wipes, but these methods are inefficient and often recover the samples into relatively large liquid volumes. Poor recoveries and large sample volumes result in samples that are often too dilute to allow for rapid detection. Further, it is desirable to be able to perform sample recovery in a field setting with minimal equipment and minimal user steps, but these conventional methods frequently require significant user steps and a laboratory setting where vortexers or other AC powered laboratory equipment are available.

### SUMMARY OF THE SUBJECT DISCLOSURE

The present disclosure addresses the problem outlined and advances the art by providing a highly efficient and easy to use, single-use surface sampling device for collection of biological and other particle types from large surface areas. The collected particles are then eluted by the user into a small liquid volume for subsequent analysis.

The subject disclosure is applicable to any field of human clinical, veterinary, food safety, pharmaceutical, outbreak investigations, forensics, biodefense and bioterrorism response, environmental monitoring, and other applications where collection of samples from surfaces and humans or animals is required. Swab and wipe sampling is routinely used in these and other fields to collect surface samples, human clinical, animal clinical and other sample types. More particularly, the subject disclosure utilizes devices, systems, and methods for sampling of large surface areas for biological particles and extraction of the collected biological particles into a small liquid volume for subsequent analysis.

In one exemplary embodiment, the present subject disclosure is a single use sampling device used to collect a sample from a surface by method of manual wiping and subsequently serve as the primary transport container and a sample extraction device. A foam sponge sampling material is bonded to the inside of a bag, which includes features or methods of being sealed and creating a liquid and airtight barrier. Additionally, installed on the bottom end of the bag is a sample access port for removing the eluted sample. In some configurations the bag may not include a sample access port, but rather the user may use a serological pipette tip or other device to reach into the bag and remove the eluted sample.

For sampling, the bag is inverted, such that the sampling material is on the outside of the bag and the user places a hand into the bag. Sampling is performed by wiping a surface with the sampling material while the user's hand is inside the bag. After sampling, the bag is turned right side out with the sampling material inside the bag. The sampling material is either wetted prior to sampling and the residual liquid is used for extraction, or an extraction buffer is added to the bag prior to sealing.

To elute collected material from the sampling material the user compresses and releases the sampling material by pressing on the sides of the bag or the user massages the sampling material through the bag walls. The user then compresses the material, through the bag walls, while holding the bag vertically so that the released liquid sample drains to the sample access port. A syringe or other method of extracting the sample is then used.

In one exemplary embodiment, the foam sponge sampling material is pre-wetted during manufacturing and then sealed within an enclosure formed on one side by the inside bag wall, or a reinforcing layer bonded to the bag wall, and on the other sides by a plastic cup with a pull handle. In this case the device is used as described above, however the user first removes the plastic cup from the sampling material by pulling on the pull handle. The sampling and extraction liquid is then present in the foam sampling material.

In another exemplary embodiment, multiple sampling bags are inside of each other or next to each other and held together by a clip. The bags are then sealed inside of an outside sealed bag. The user can open said outside bag and attach it to a belt or other device for easy access to multiple sampling bags.

An exemplary embodiment was used in a laboratory study to determine the recovery efficiency for bacterial endospores from stainless steel coupons. Large area surface samplers with three selected collection materials were tested for their ability to collect endospores. A Barcode Bacillus thuringiensis kurstaki (Btk) spore prep was used as a surrogate for Bacillus anthracis. All surface testing was done with a 1 mL spike of 1×10⁴ CFU/mL spread dropwise onto the test coupons which was allowed to dry in a biological cabinet for up to 30 minutes. Coupons with two different surface types, electro-polished stainless steel and stainless steel with a 2B surface finish, were used. Between tests, the stainless steel coupons were disinfected by adding a 10% bleach solution onto the plates for 5 minutes, which was wiped off and cleaned with DI water, followed by spraying and wiping with 70% ethanol. The coupons then sat in a biological cabinet, under UV light, for at least an hour prior to spiking with spores.

The results, as shown in Table 1, demonstrated that a 100 PPI polyester polyurethane foam material candidate provided improved recovery compared to an ultra-fine polyester polyurethane and a melamine foam.

TABLE 1
Average Recovery of Btk Spores from two Types of Stainless Steel with
Selected Foam Materials and two Wetting agents, and stainless coupons
Recovery of Spores from Two Surfaces with Three Foam Types &
Two Buffers

| | Electropolished Stainless Steel | | 2B Stainless |
|---|---|---|---|
| | PBS w/0.1% | | PBS w/0.1% |
| Foam Material | Tween 20 | PBS only | Tween 20 |
| 100 PPI polyester | 67.9% | 37.7% | 52.4% |
| polyurethane | | | |
| Ultrafine polyester | 53.4% | 25.8% | 34.2% |
| polyurethane | | | |
| Melamine | 66.0% | 16.7% | 23.4% |

### BRIEF DESCRIPTION OF THE DRAWINGS

FIG. 1A shows a surface sampling method for an exemplary embodiment of a large surface area sampler with Surface Sampling Step 1—Use of an overlapping ‘S’ pattern to cover the entire surface with horizontal strokes, according to an exemplary embodiment of the present subject disclosure.

FIG. 1B shows a surface sampling method for an exemplary embodiment of a large surface area sampler with Surface Sampling Step 2—Use of an overlapping ‘S’ pattern to cover the entire surface with vertical strokes, according to an exemplary embodiment of the present subject disclosure.

FIG. 1C shows a surface sampling method for an exemplary embodiment of a large surface area sampler with Surface Sampling Step 3—Use of an overlapping ‘S’ pattern to cover the entire surface with diagonal strokes, according to an exemplary embodiment of the present subject disclosure.

FIG. 2A shows a top view of an exemplary embodiment of a large area surface sampler in the manufactured state, according to an exemplary embodiment of the present subject disclosure.

FIG. 2B shows a bottom view of an exemplary embodiment of a large area surface sampler in the manufactured state, according to an exemplary embodiment of the present subject disclosure.

FIG. 3A shows a top view of a large area surface sampler after removing the protective cup that covers the sampling material, according to an exemplary embodiment of the present subject disclosure.

FIG. 3B shows a bottom view of a large area surface sampler after removing the protective cup that covers the sampling material, according to an exemplary embodiment of the present subject disclosure.

FIG. 4 shows a large area surface sampler after extraction of the sampling material and prior to withdrawing the liquid sample, according to an exemplary embodiment of the present subject disclosure.

FIG. 5 shows a large area surface sampler in the manufactured state with an inverted bag cuff, according to an exemplary embodiment of the present subject disclosure.

FIG. 6 shows a perspective view, from the angle of the bag opening, of a large area surface sampler in the manufactured state with an inverted bag cuff, according to an exemplary embodiment of the present subject disclosure.

FIG. 7 shows a perspective view, from the angle of the bag opening, of a large area surface sampler after removing the protective cup that covers the sampling material, according to an exemplary embodiment of the present subject disclosure.

FIG. 8 shows a sampling package component of a large area surface sampler, according to an exemplary embodiment of the present subject disclosure.

FIG. 9 shows an exploded view of a sampling package component of a large area surface sampler, according to an exemplary embodiment of the present subject disclosure.

### DETAILED DESCRIPTION OF THE SUBJECT DISCLOSURE

The present subject disclosure describes devices, systems, and methods relating to a single use large area surface sampler with invertible bag used to collect a sample from a surface by method of manual wiping and which subsequently serves as a containment and extraction device. Further the invertible bag can act as a primary transport container during sample transport. The device is capable of collection from large areas, generally regarded as up to 1 m². The device is also designed to be functional and easy to use while in proper Personal Protective Equipment (PPE) such as Level A or MOPP gear Level 4.

The basic construction of the large area surface sampler with invertible bag is that of a piece of sampling material rigidly connected to the inside of a bag. The bag is provided to the user in an inverted state and the user places their hand into the bag—allowing the bag to be used as a contaminant barrier during sampling. The user can place their hand into the bag with their palm facing the sampling material or facing away from the sampling material.

After sampling a surface, the bag is then reinverted and sealed, creating a liquid and airtight extraction device and primary transport container. Additionally, installed on the bag is a bag access port which is to be permanently affixed to the bottom of the bag. This port allows for the sample to quickly and easily be extracted from the bag upon arrival at the testing facility.

Further, the sampling material is sealed inside of a protective cup that reduces the potential for contamination and allows a sampling and extraction fluid to be sealed in the sampling material during manufacturing. During use the user pulls on a handle on the protective cup, exposing the prewetted sampling material. After sampling the bag is re-inverted, as described above, and the sampling material is compressed multiple times to release the captured material. Finally, the sampling material is compressed fully while holding the bag vertically, such that the liquid sample drains to the sampling port and can be removed using a syringe or other device through a self-opening valve.

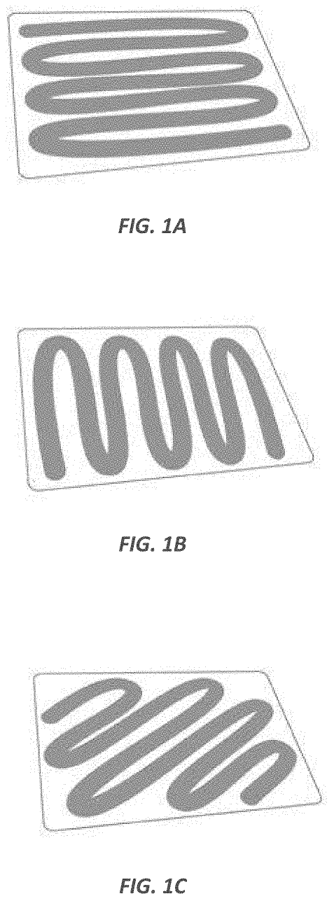

The basic function of the large area surface sampler with invertible bag is to serve as an easy-to-use device to collect a biological sample from a surface while encumbered by mobility restrictive equipment. To accomplish this the large area surface sampler with invertible bag will be provided to the operator in the sampling orientation with the bag turned inside out. The operator slides their gloved hand into the flexible polyethylene bag in the sampling orientation until there is sufficient friction to keep the bag in place during sampling. The surface sampling is generally completed using CDC or similar guidelines as shown in FIGS. 1A, 1B, and 1C. FIG. 1A shows Surface Sampling Step 1—Use an overlapping ‘S’ pattern to cover the entire surface with horizontal strokes. FIG. 1B shows Surface Sampling Step 2—Use an overlapping ‘S’ pattern to cover the entire surface with vertical strokes. FIG. 1C shows Surface Sampling Step 3—Use an overlapping ‘S’ pattern to cover the entire surface with diagonal strokes.

After collection, the base of the bag can be held as an anchor by the sampling hand while the other hand grabs the closure of the bag and pulls the bag right side out by moving both hands past each other. Once inverted, the bag can be sealed and will serve as the primary transport container and can be used for sample extraction in the field. Per federal regulations, both the primary and secondary containment vessels must be airtight and liquid tight for the shipment of Category A infectious substances. With a proper leak proof closure, the bag serves as a primary container. The advantage here is that the sample does not need to be extracted while still in the field and can instead be transported to a laboratory and extracted when desired. This can mean reducing the amount of time required to collect a sample and ultimately the time an operator spends at a sampling location.

Various methods of sample collection from surfaces already exist. Many of these methods involve swabs which can only collect from relatively small areas, 25 cm² or 1 ft². The large area surface sampler with invertible bag is equipped with a hydrophobic polyester, polyurethane foam sampling material with increased size compared to traditional swabs and samplers. The larger material allows for sampling of surfaces greater than 1 ft².

In addition to the hydrophobic polyester, polyurethane foam sampling material, many other types of sampling materials, that are well known to and appreciated by those skilled in the art, can be used including alternative polyurethanes, hydrophilic polyurethanes, polyether polyurethanes, cellulose sponge, melamine foam, rayon, cotton, and other foam and filter materials. The polyurethane material in the present example was selected for its resistance to breaking down during sampling, ability to hold fluid during transport, recovery efficiency of organisms, and medical grade.

Current state-of-the-art surface samplers typically have fluid which is separate from the sampling material. The fluid is then required to be added to the sponge for wet sampling. This requires extra steps to be performed which can cause significant increases in the amount of time it takes to collect the sample, especially when utilizing a hydrophobic sampling material. As the large area surface sampler with invertible bag utilizes a hydrophobic sampling material, which is challenging and time consuming to wet while in the field, it will be provided in one of two configurations: dry or pre-wetted. Pre-wetted Mano will contain from 1 mL to 100 mL, but more commonly 10 to 25 mL of sampling fluid.

For the large area surface sampler with invertible bag to be provided pre-wetted, a sampling and extraction fluid is added to the sampling material prior to sealing it into a protective air-tight cup. The protective cup can be made from a range of materials that will be well appreciated by those skilled in the art, including, but not limited to: injection molded thermoplastic, formed thermoplastic, flexible plastic films, and flexible rubber films, and the like.

The self-contained sampling device that is bonded to the sampling bag to form the large area surface sampler with invertible bag is referred to as the sampling package component or sampling package or simply the package. The package is constructed of four components. The top piece in the figure is the hard-plastic cup, or cover, followed by the sampling material. These pieces are both individually heat sealed by an induction heat sealer onto the next piece shown, a foil material with a heat-activated peel-able adhesive. The heat seal is designed to hold tight in extreme conditions, such as altitude up to 20,000 feet, but to release easily when the cup handle is pulled by the user. To achieve this, the heat seal width is narrow and generally the heat seal is in a multi-sided shape around the edge of the cup in the sealing area. The multi-sided seal may be any shape, such as a decagon, octagon, or hexagon shape. This multisided seal, or a seal with a point at the starting point of the release during opening, enables the seal to be more easily opened when compared to a circular seal. Further, during the sealing process, the cup may be compressed to remove excess gas so that during high altitude transport, expansion of the gas is limited and as such undue stress is not placed on the seal. The last piece of the seal is a double-sided adhesive tape. This tape is used to join the foil backing to the sampling bag.

The package is constructed such that when applied to the bag with the double-sided adhesive, the cover can be peeled or separated from the foil backing material without separation of the bag from the package. This is made possible by the handle extending from the cup which is not welded onto the bag. When the cover is peeled away, the double-sided adhesive, the foil and the sampling material remain permanently affixed to the inside of the bag. This orientation with the sampling material exposed and facing out allows for the collection of the sample.

The bag sample access port allows the sample to be extracted from the bag without the need for a pipette or pipette tip. The use of such a device can be difficult when using such a large bag. Also, it could result in the entire Pipette tip becoming contaminated. Lastly, it requires the bag holding the sample to be open along the entire width of the bag. Instead, a bag access port allows a syringe device, or any device with a Luer style connection to remove the sample easily and quickly from the bag in order to minimize any potential contamination or risk to the operator that would be created by alternative methods.

As the sampling material is on the inside of the bag, the bag needs to be openable in order to expose the sampling material to the sampling surface. This requires the use of a closure device with the capability of, at a minimum, closed after sampling. Generally, methods like heat sealing which would allow the bag to be sealed once in the transportation position are not conducive to the type of sampling being performed as they require equipment that is large and bulky in order to create consistent leakproof seals. Other methods are possible in order to find something which could create a leakproof seal. Adhesives which can fold over and create a seal as seen with 95 KPa bags require an extra tab of material at the top of the bag which would make inversion of the bag difficult and would risk compromising the integrity of the bag. Wire tab seal methods that are used with “Whirl-Pak” bags may also be used. Some exemplary embodiments use a press to seal closure device which is capable of creating an air-tight and leak-free seal. This type of seal is extremely effective as it is easy to do while in MOPP gear and also the bag to be reopened as needed. It is recommended that a layer of tape be used on the sealed and folded over closure device to ensure as a secondary protective measure that the bag is unable to open itself by any means.

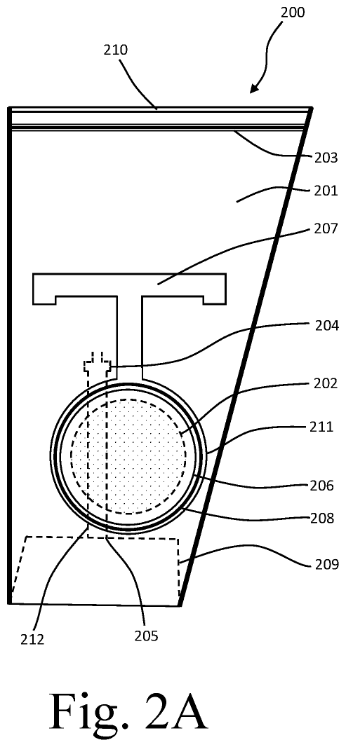

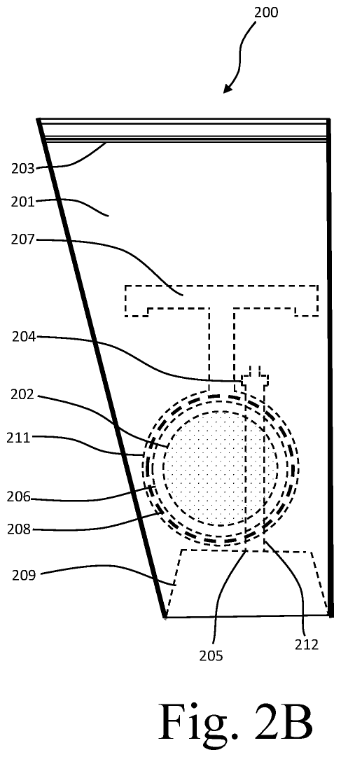

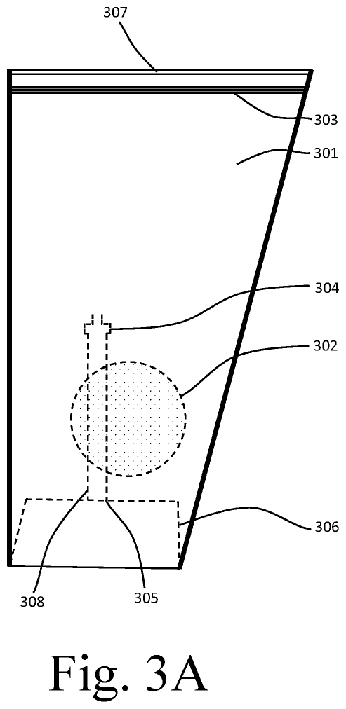

FIGS. 2A and 2B show an exemplary embodiment of a large area surface sampler 200 in the manufactured state. In the manufactured state—prior to use—the bag 201 has sampling material 202, bonded to the inner bag 201 surface and leak proof closure 203. The leak proof closure 203 can be selected from a range of options that will be known to and appreciated by those skilled in the art, including but not limited to: zipper-type closures, folded waterproof bag type seals, adhesive seals, and twisted seals. Leak proof closure 203 is used to seal bag 201 at opening 210. Sampling material 202 can be selected from a range of materials that will be known to and appreciated by those skilled in the art, including but not limited to: foam sponge materials; filter materials including, but not limited to: non-woven, woven, membrane, depth, electret, hydrophobic, hydrophilic filter materials. The sampling material may be packaged in dry or wet state during manufacturing. The wetting agent can be selected from a range of liquids that will be known to and appreciated by those skilled in the art, including but not limited to: water, surfactant solutions, buffers, and solvents along with other additives such as neutralizing agents. Further the wetting agent can be designed to maintain bacterial or other biological particle viability, or it may be designed to lyse these materials during the extraction process. Specific additives to the wetting agent or collection and extraction fluid, can be selected from a range of liquids that will be known to and appreciated by those skilled in the art, including but not limited to: phosphate buffered saline; tris buffer; or other buffers; EDTA, viral transport medial, growth media, beef extract buffer, or surfactants including polysorbate 20, polysorbate 80, Triton X-100, sodium dodecyl sulfate, polaxamer 184, Pluronic, and other ionic, nonionic, cationic and zwitterionic surfactants or proteins or growth media. Tween 20 has been used extensively in the disclosed large area surface sampler with effective results, but in some cases is not compatible with some viability based assays, such as TCID50. In these cases, a surfactant such as Pluronic and media such as beef extract buffer have been demonstrated to provide efficient recovery from the surface and the collection material and to be compatible with viability assays. A range of other buffers may be added to maintain the elution fluid pH in a specific range or to supplement the fluid in other ways, such as maintain a specific ionic strength. Further, use of lysis fluids for extraction and lysis of capture microbes or inclusion of assay specific components can be used.

The sampling material 202 can be bonded to the bag using any number of methods and techniques that will be appreciated by those skilled in the art, including but not limited to: tapes and adhesive films, contact adhesives, spray adhesives, thermal adhesives, pressure-sensitive adhesives, vibration welding, ultrasonic welding, and thermal welding. The sampling bag 201 can include a sampling port 204 which is non-removably bonded to flexible tube 212 which is attached to bag 201 at seal 205, which is a non-removable seal. The sampling port 204 can be selected from any number of devices that will be appreciated by those skilled in the art, including but not limited to: self-opening valves, luer-lock valves, self-opening luer lock valves, self-sealing ports, septums, split septums, self-healing ports, spike ports, push-pull connectors, John Guest connectors, and push-to-connect. Further, sampling port 204 can simply be a clamp that is placed over flexible tube 212.

The sampling material 202 can be left uncovered if desired or may be covered by a protective cup 206. FIG. 2A shows a top view of a large area surface sampler 200 with sampling material 202 and protective cup 206, and FIG. 2B shows a bottom view of a large area surface sampler 200 with sampling material 202 and protective cup 206. If the sampling material 202 is covered, then it may be first filled with a wetting agent prior to sealing the protective cup 206 against the bag 201. The protective cup 206 may be of a thermoplastic or other materials that will be known to and appreciated by those skilled in the art. The protective cup 206 can be sealed to the bag 201 using seal 208, which is a removable seal, which can be selected from any number of options that will be known to and appreciated by those skilled in the art, including but not limited to: tapes and adhesive films, contact adhesives, spray adhesives, thermal adhesives, pressure-sensitive adhesives, vibration welding, ultrasonic welding, and thermal welding. Protective cup 206 includes handle 207 which is used to remove the protective cup 206 from bag 201 prior to sampling. Further protective cup 206 includes sealing flange 211 which allows for bonding to bag 201.

Bag 201 is provided to users in the inverted state. In this configuration the sampling material 202 is on the outside of the inverted bag 201. The bottom 209 of bag 201 can be left sticking into the inside of the bag 201. This is shown in FIG. 2A from the top side view with sampling material 202 and protective cup 206 viewed through both layers of the bag 201 and with sampling port 204 and seal 205 viewed through the top layer of the bag 201. FIG. 2B shows the bottom side view of bag 201 with sampling material 202 and protective cup 206 on the outside of the bag 201.

To use the large area surface sampler 200, the user places their hand into bag 201 through the opening at leak proof closure 203. The user may use the large area surface sampler 200 with the palm of the hand facing toward or away from sampling material 202. While keeping one hand in the bag 201, the user grasps handle 207 with their second hand and peels it from bag 201.

While leaving their hand in the bag 201 the user can then wipe a surface with the sampling material 202 using any number of methods including linear passes followed by passes at 90 degrees and 45 degrees to the first pass as is outlined in the CDC sampling method, circular motion or other motions that will be known to and appreciated by those skilled in the art.

After sampling the user can grasp the bottom 209 of bag 201 with their hand in the bag and using the second hand they can grasp at or near leak proof closure 203. By pulling with both hands towards each other—until passing each other—the user can then reinvert the bag 201. To make grasping and reinversion of the bag 201 easier for the user, the bag 201, near leak proof closure 203 can be folded over, forming a cuff. In this way inversion of bag 201 is already initiated. Further, the user can grasp flexible tube 212 or flexible tube 212 and bottom 209 together to assist in the inversion process. A removable rigid or flexible cap can also be provided over sampling port 204 to reduce the potential for contamination of sampling port 204. The cap is removed from sampling port 204 before extract of sample from sampling port 204.

Bag 201 can then be either transported to a laboratory or other location for extraction and analysis or extraction can be performed on site. For transport, bag 201 is sealed using leak proof closure 203 and placed inside of a secondary container prior to transport. For extraction of a dry sampling material 202, a liquid extraction buffer is added to the inside of the bag and the leak proof closure 203 is closed. For extraction of a wet sampling material 202, the leak proof closure 203 is simply closed.

To extract the sampling material 203, the user holds bag 201 horizontally with sampling material 202 at the bottom of the bag. The bag 201 is then folded until the leak proof closure 203 end and the sampling port 204 end are touching or nearly touching. In this way sampling material 202 is at the bottom of the now U-shaped bag 201. The user then grasps sampling material 202 in the palm of one hand, through the wall of bag 201, and tightly squeezes the sampling material 202—expelling most of the liquid sample out of the sampling material 202. The sampling material 202 is then released so that the liquid flows back out of said sampling material. Continuing to hold bag 201 in the same configuration and orientation the user then repeats the squeeze and release method at least two more times.

Various methods of compressing or massaging the sampling material 202 through the wall of bag 201 to extract the liquid sample and collected material can be used including, but not limited to: massaging with one had on each side of bag 201 being moved in a circular motion while compressing sampling material 202, by laying the bag on a surface and then pressing down with a hand onto the sampling material 202 through the wall of bag 201, by passing bag 201 through compression rollers, by lay bag 201 on a surface and then passing a roller over the bag 201 or by pressing down on bag with a flat material, by twisting bag 201 to “wring” out the sampling material 202, and any other technique by which the sampling material can be compressed or massaged through the wall of bag 201 without damaging bag 201. Massaging or compression of sampling material 202 can be performed using any number of methods and techniques that will be appreciated by those skilled in the art,

The user then holds bag 201 vertically such that leak proof closure 203 is at the top of the bag 201 and the sampling port 204 is at the bottom of the bag 201. While holding bag 201 in this configuration and orientation, the user then tightly squeezes the sampling material 202 in the palm of one hand until nearly all of the liquid sample has been expelled. The user continues to hold bag 201 vertically until nearly all liquid sample has drained to sampling port 204. Again, continuing to hold bag 201 vertically the user now attaches a syringe or other sample extraction device to sampling port 204. The user then pulls back on the syringe barrel until nearly all of the liquid sampling has been withdrawn from bag 201.

Other methods of extraction, that are like the method described above or three repeated compress and release steps performed in the palm of the hand, can also be used with the large area surface sampler 200. These include the following: compressing and releasing the sampling material 202, through the wall of bag 201, between the palms of two hands; massaging the sampling material by circular and up and down compression and movement; and placing the bag on a flat surface or in a bowl or other cupped surface and then compressing and releasing or massaging with one or two hands.

After removal of the liquid sample from bag 201, the user or laboratory may utilize any number of analysis methods that will be known to and appreciated by those skilled in the art including, but not limited to the following: culture/enumerate, polymerase chain reaction, quantitative real-time polymerase chain reaction, ATP-based methods, sequencing, antigen assays, biochemical assays, molecular detection methods, other rapid microbiological detection methods, mass spectrometry, chemical, cytometry, flow cytometry, or other growth-based microbiology methods to detect biological particles. Additionally, chemical analysis techniques may be used on the extracted sample to detect and identify particles of biological or non-biological nature.

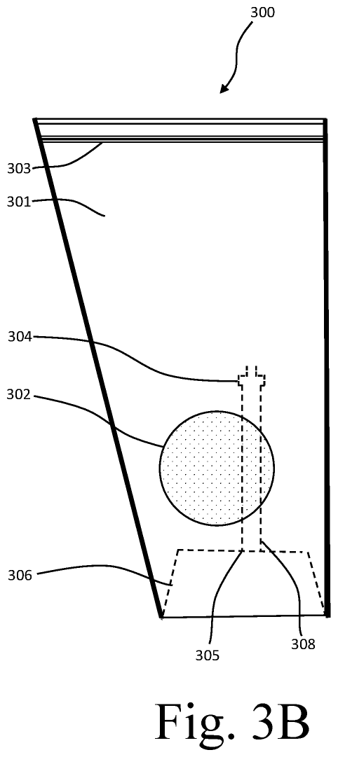

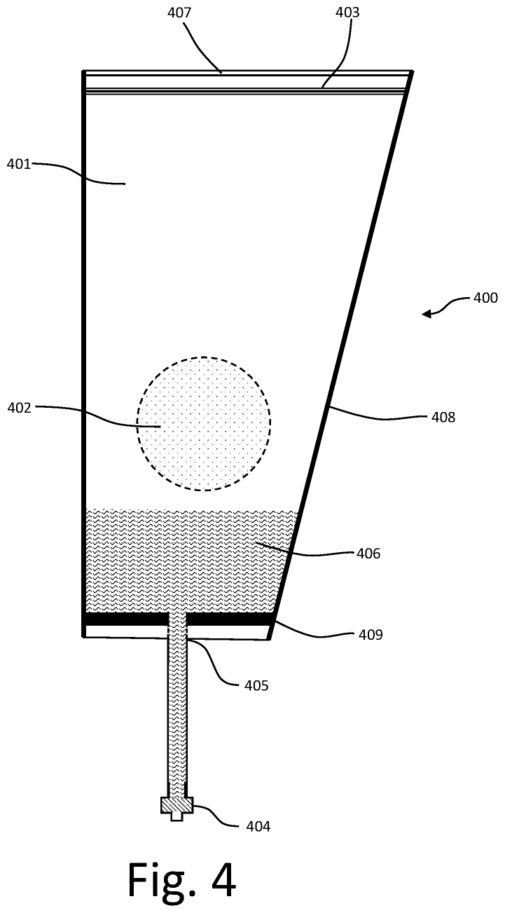

FIGS. 3A and 3B show an exemplary embodiment of a large area surface sampler 300 without a protective cup covering the sampling material 302. In the manufactured state—prior to use—the bag 301 has sampling material 302, bonded to the inner bag 301 surface and leak proof closure 303. The leak proof closure 303 can be selected from a range of options that will be known to and appreciated by those skilled in the art, including but not limited to: zipper-type closures, folded waterproof bag type seals, adhesive seals, and twisted seals. Sampling material 302 can be selected from a range of materials that will be known to and appreciated by those skilled in the art, including but not limited to: foam sponge materials; filter materials including, but not limited to: non-woven, woven, membrane, depth, electret, hydrophobic, hydrophilic filter materials. The sampling material may be packaged in dry or wet state during manufacturing. The wetting agent can be selected from a range of liquids that will be known to and appreciated by those skilled in the art, including but not limited to: water, surfactant solutions, buffers, and solvents along with other additives such as neutralizing agents.

The sampling material 302 can be bonded to the bag using any number of techniques that will be appreciated by known to and appreciated by those skilled in the art, including but not limited to: tapes and adhesive films, contact adhesives, spray adhesives, thermal adhesives, pressure-sensitive adhesives, vibration welding, ultrasonic welding, and thermal welding. The sampling bag 301 can include a sampling port 304 which is attached flexible tube 308 and then to bag 301 at seal 305. The sampling port 304 can be selected from any number of devices that will be appreciated by those skilled in the art, including but not limited to: self-opening valves, luer-lock valves, self-opening luer lock valves, self-sealing ports, septums, split septums, self-healing ports, spike ports, push-pull connectors, John Guest connectors, and push-to-connect.

The sampling material 302 can be left uncovered if desired, as is shown in FIGS. 3A and 3B, or may be covered by a protective cup. If the sampling material 302 is covered, then it may be first filled with a wetting agent prior to sealing a protective cup over the sampling material 302 and against the bag 301.

FIGS. 3A and 3B are provided here to show the state of the bag 301 after removal of the protective cup, or as manufactured without a protective cup. FIG. 3A shows a top view of a large area surface sampler 300 with sampling material 302 under bag 301. FIG. 3B shows a bottom view of a large area surface sampler 300 with bag 301 flipped over so that sampling material 302 is on top. Bag 301 is used in the same manner as described above for bag 201 in FIGS. 2A and 2B and will not be repeated again here for sake of brevity

FIG. 4 shows an exemplary embodiment of a large area surface sampler 400 after reinversion of bag 401 and extraction of a liquid sample 406 from the sampling material 402. The bag 401 has sampling material 402, bonded to the inner bag 401 surface and leak proof closure 403. The leak proof closure 403 can be selected from a range of options that will be known to and appreciated by those skilled in the art, including but not limited to: zipper-type closures, folded waterproof bag type seals, adhesive seals, and twisted seals. Sampling material 402 can be selected from a range of materials that will be known to and appreciated by those skilled in the art, including but not limited to: foam sponge materials; filter materials including, but not limited to: non-woven, woven, membrane, depth, electret, hydrophobic, hydrophilic filter materials. The sampling material may be packaged in dry or wet state during manufacturing. The wetting agent can be selected from a range of liquids that will be known to and appreciated by those skilled in the art, including but not limited to: water, surfactant solutions, buffers, and solvents along with other additives such as neutralizing agents.

The sampling material 402 can be bonded to the bag using any number of techniques that will be appreciated by those skilled in the art, including but not limited to: tapes and adhesive films, contact adhesives, spray adhesives, thermal adhesives, pressure-sensitive adhesives, vibration welding, ultrasonic welding, and thermal welding. The sampling bag 401 can include a sampling port 404 which is attached to bag 401 at seal 405. The sampling port 404 can be selected from any number of devices that will be appreciated by those skilled in the art, including but not limited to: self-opening valves, luer-lock valves, self-opening luer lock valves, self-sealing ports, septums, split septums, self-healing ports, spike ports, push-pull connectors, John Guest connectors, and push-to-connect.

FIG. 4 is provided here to primarily show the state of the bag 401 after extraction of sampling material 402. Bag 401 used is the same as described above for bag 201 in FIG. 2A and FIG. 2B. After extraction of sampling material 402, the liquid sample 406 is allowed to drain to the bottom of bag 401. The user may then attach a sample extraction device, such as a luer lock syringe to sampling port 404 and remove the sample from bag 401 by drawing back on the syringe plunger.

After removal of the liquid sample from bag 401, the user or laboratory may utilize any number of analysis methods that will be known to and appreciated by those skilled in the art including, but not limited to the following: culture/enumerate, polymerase chain reaction, quantitative real-time polymerase chain reaction, ATP-based methods, sequencing, antigen assays, biochemical assays, molecular detection methods, other rapid microbiological detection methods, mass spectrometry, chemical, cytometry, flow cytometry, or other growth-based microbiology methods to detect biological particles. Additionally, chemical analysis techniques may be used on the extracted sample to detect and identify particles of biological or non-biological nature.

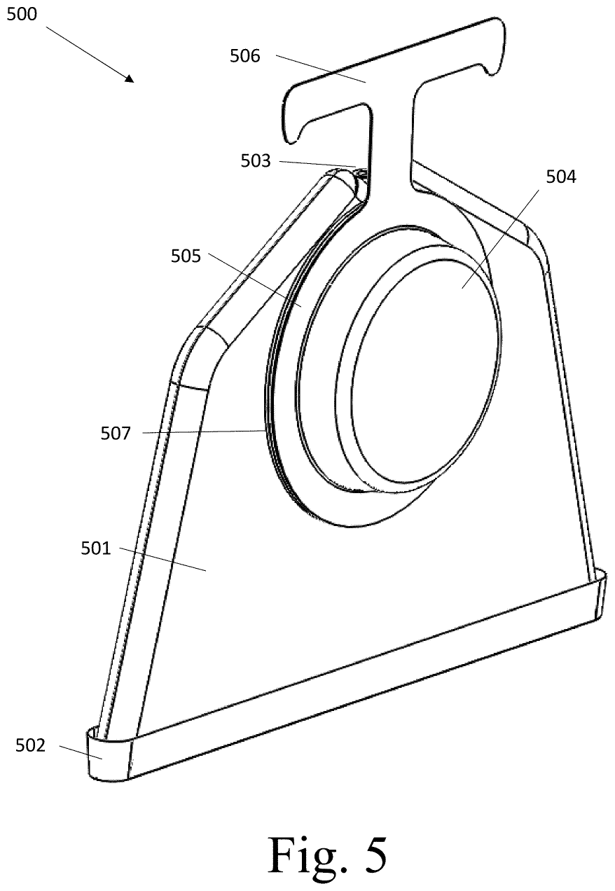

FIG. 5 show an exemplary embodiment of a large area surface sampler 500 in the manufactured state. In the manufactured state—prior to use—the bag 501 has sampling material bonded to the inner bag 501 surface and leak proof closure 502. The leak proof closure 502 can be selected from a range of options that will be known to and appreciated by those skilled in the art, including but not limited to: zipper-type closures, folded waterproof bag type seals, adhesive seals, and twisted seals. The sampling material is not visible in this drawing because it is covered by the protective cup 504 with flat sealing ring 505 and handle 506. The sampling material can be selected from a range of materials that will be known to and appreciated by those skilled in the art, including but not limited to: foam sponge materials; filter materials including, but not limited to: non-woven, woven, membrane, depth, electret, hydrophobic, hydrophilic filter materials. The sampling material may be packaged in dry or wet state during manufacturing. The wetting agent can be selected from a range of liquids that will be known to and appreciated by those skilled in the art, including but not limited to: water, surfactant solutions, buffers, and solvents along with other additives such as neutralizing agents.

The sampling material can be bonded to the bag using any number of methods that will be known to and appreciated by those skilled in the art, including but not limited to: tapes and adhesive films, contact adhesives, spray adhesives, thermal adhesives, pressure-sensitive adhesives, vibration welding, ultrasonic welding, and thermal welding. The sampling bag 501 can include a sampling port 503 which is attached to bag 501. The sampling port 503 can be selected from any number of devices that will be known to and appreciated by those skilled in the art, including but not limited to: self-opening valves, luer-lock valves, self-opening luer lock valves, self-sealing ports, septums, split septums, self-healing ports, spike ports, push-pull connectors, John Guest connectors, and push-to-connect.

The sampling material can be left uncovered if desired or may be covered, as is shown in FIG. 5, by a protective cup 504. If the sampling material is covered, then it may be first filled with a wetting agent prior to sealing the protective cup 504 against the bag 501. The protective cup 504 may be of a thermoplastic or other materials that will be known to and appreciated by those skilled in the art. The protective cup 504 can be sealed to the bag 501 using seal 507 between the cup sealing ring 505 and the bag 501. The seal 507 is a non-permanent, removable seal, which can be selected from any number of methods that will be known to those skilled in the art, including but not limited to: tapes and adhesive films, contact adhesives, spray adhesives, thermal adhesives, pressure-sensitive adhesives, vibration welding, ultrasonic welding, and thermal welding. Protective cup 504 includes handle 506 which is used to remove the protective cup 504 from bag 501 prior to sampling.

Bag 501 is provided to users in the inverted state. In this configuration the sampling material is on the outside of the inverted bag 501. The top of the bag 501 and leak proof closure 502 are left folded over as shown so that the user can grasp this material when reinverting the bag after sampling and before extraction. The bottom of the bag 501, near sampling port 503, can be left sticking into the inside of the bag 501, for another point for the user to grab when reinverting the bag 501.

To use the large area surface sampler 500, the user places their hand into bag 501 through the opening at leak proof closure 502. The user may use the large area surface sampler 500 with the palm of the hand facing toward or away from sampling material and protective cup 504. While keeping one hand in the bag 501, the user grasps handle 506 with their second hand and peels it from bag 501, exposing the sampling material.

While leaving their hand in the bag 501 the user can then wipe a surface with the sampling material using any number of methods including linear passes followed by passes at 90 degrees and 45 degrees to the first pass as is outlined in the CDC sampling method, circular motion or other motions that will be known to and appreciated by those skilled in the art.

After sampling, the user can grasp the inside bottom of bag 501 with their hand in the bag and, using the second hand, they can grasp the folded over bag material near the leak proof closure 502. By pulling with both hands towards each other—until passing each other—the user can then reinvert the bag 501.

Bag 501 can then be either transported to a laboratory or other location for extraction and analysis or extraction can be performed on site. For transport, bag 501 is sealed using leak proof closure 502 and placed inside of a secondary container prior to transport. For extraction of a dry sampling material, a liquid extraction buffer is added to the inside of the bag and the leak proof closure 502 is closed. For extraction of a wet sampling material, the leak proof closure 502 is simply closed prior to extraction.

To extract the sampling material, the user holds bag 501 horizontally with sampling material at the bottom of the bag. The bag 501 is then folded until the leak proof closure 502 end and the sampling port 503 end are touching or nearly touching. In this way sampling material is at the bottom of the now U-shaped bag 501. The user then grasps the sampling material in the palm of one hand, through the wall of bag 501, and tightly squeezes the sampling material—expelling most of the liquid sample out of the sampling material. The sampling material is then released so that the liquid flows back out of said sampling material. Continuing to hold bag 501 in the same configuration and orientation the user then repeats the squeeze and release method at least two more times.

The user then holds bag 501 vertically such that leak proof closure 502 is at the top of the bag 501 and the sampling port 503 is at the bottom of the bag 501. While holding bag 501 in this configuration and orientation the user then tightly squeezes the sampling material in the palm of one hand until nearly all of the liquid sample has been expelled. The user continues to hold bag 501 vertically until nearly all liquid sample has drained to sampling port 503. Again, continuing to hold bag 501 vertically the user now attaches a syringe or other sample extraction device to sampling port 503. The user then pulls back on the syringe barrel until nearly all of the liquid sampling has been withdrawn from bag 501.

Other methods of extraction, that are like the method described above or three repeated compress and release steps performed in the palm of the hand, can also be used with the large area surface sampler 500. These include the following: compressing and releasing the sampling material, through the wall of bag 501, between the palms of two hands; massaging the sampling material by circular and up and down compression and movement; and placing the bag 501 on a flat surface or in a bowl or other cupped surface and then compressing and releasing or massaging with one or two hands.

After removal of the liquid sample from bag 501, the user or laboratory may utilize any number of analysis methods that will be known to and appreciated by those skilled in the art including, but not limited to the following: culture/enumerate, polymerase chain reaction, quantitative real-time polymerase chain reaction, ATP-based methods, sequencing, antigen assays, biochemical assays, molecular detection methods, other rapid microbiological detection methods, mass spectrometry, chemical, cytometry, flow cytometry, or other growth-based microbiology methods to detect biological particles. Additionally, chemical analysis techniques may be used on the extracted sample to detect and identify particles of biological or non-biological nature.

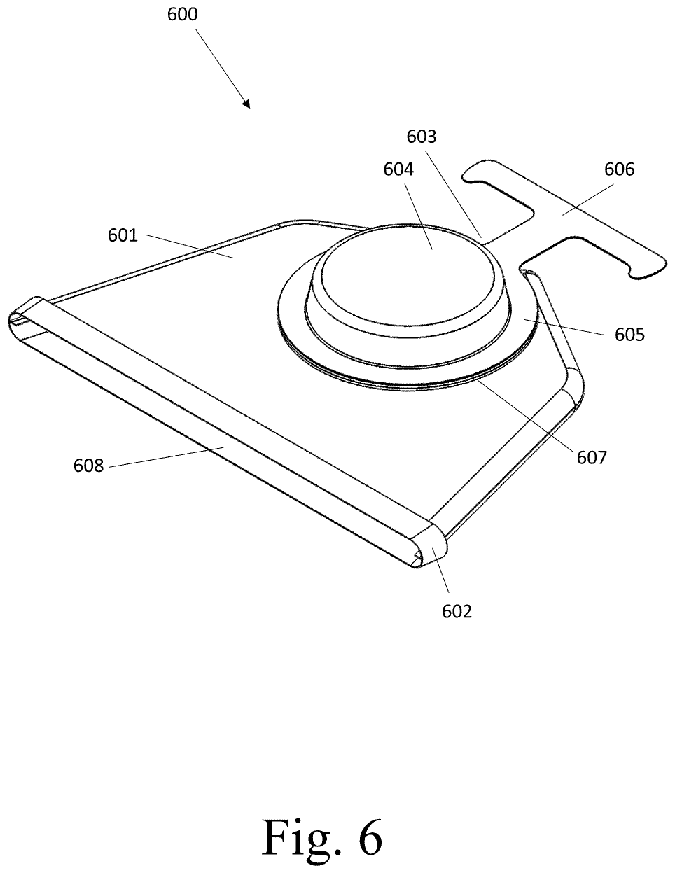

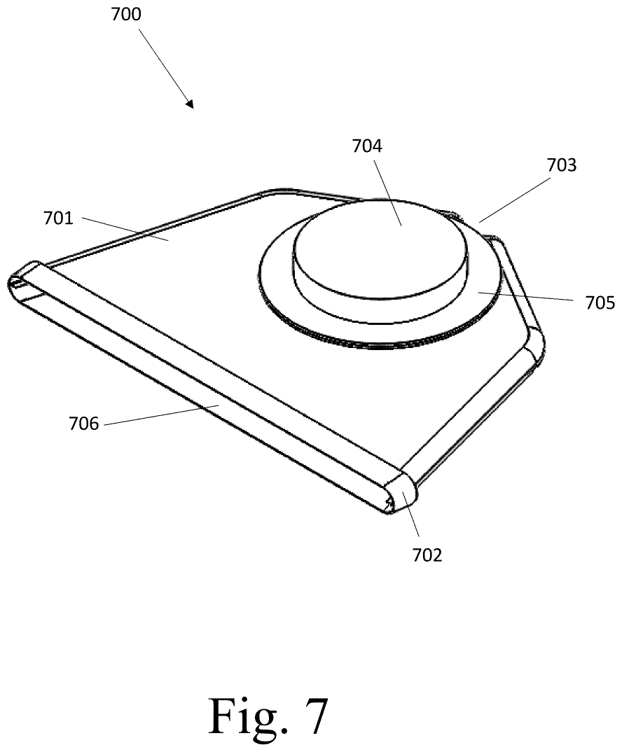

FIG. 6 show an exemplary embodiment of a large area surface sampler 600 in the manufactured state. In the manufactured state—prior to use—the bag 601 has sampling material bonded to the inner bag 601 surface with leak proof closure 602 and opening 608. The leak proof closure 602 can be selected from a range of methods that will be known to and appreciated by those skilled in the art, including but not limited to: zipper-type closures, folded waterproof bag type seals, adhesive seals, and twisted seals. The sampling material is not visible in this drawing because it is covered by the protective cup 604 with flat sealing ring 605 and handle 606. The sampling material can be selected from a range of materials that will be known to and appreciated by those skilled in the art, including but not limited to: foam sponge materials; filter materials including, but not limited to: non-woven, woven, membrane, depth, electret, hydrophobic, hydrophilic filter materials. The sampling material may be packaged in dry or wet state during manufacturing. The wetting agent can be selected from a range of liquids that will be known to and appreciated by those skilled in the art, including but not limited to: water, surfactant solutions, buffers, and solvents along with other additives such as neutralizing agents.

The sampling material can be bonded to the bag using any number of methods and techniques that will be appreciated by those skilled in the art, including but not limited to: tapes and adhesive films, contact adhesives, spray adhesives, thermal adhesives, pressure-sensitive adhesives, vibration welding, ultrasonic welding, and thermal welding. The sampling bag 601 can include a sampling port 603 which is attached to bag 601. The sampling port 603 can be selected from any number of devices that will be known to and appreciated by those skilled in the art, including but not limited to: self-opening valves, luer-lock valves, self-opening luer lock valves, self-sealing ports, septums, split septums, self-healing ports, spike ports, push-pull connectors, John Guest connectors, and push-to-connect.

The sampling material can be left uncovered if desired or may be covered, as is shown in FIG. 6, by a protective cup 604. If the sampling material is covered, then it may be first filled with a wetting agent prior to sealing the protective cup 604 against the bag 601. The protective cup 604 may be of a thermoplastic or other materials that will be known to and appreciated by those skilled in the art. The protective cup 604 can be sealed to the bag 601 using seal 607 between the cup sealing ring 605 and the bag 601. The seal 607 is a non-permanent, removable seal, which can be selected from any number of methods that will be known to those skilled in the art, including but not limited to: tapes and adhesive films, contact adhesives, spray adhesives, thermal adhesives, pressure-sensitive adhesives, vibration welding, ultrasonic welding, and thermal welding. Protective cup 604 includes handle 606 which is used to remove the protective cup 604 from bag 601 prior to sampling.

Bag 601 is provided to users in the inverted state. In this configuration the sampling material is on the outside of the inverted bag 601. The top of the bag 601 and leak proof closure 602 are left folded over as shown so that the user can grasp this material when reinverting the bag after sampling and before extraction. The bottom of the bag 601, near sampling port 604, can be left sticking into the inside of the bag 601, for another point for the user to grab when reinverting the bag 601.

To use the large area surface sampler 600, the user places their hand into bag 601 through the opening 608 at leak proof closure 602. The user may use the large area surface sampler 600 with the palm of the hand facing toward or away from sampling material and protective cup 604. While keeping one hand in the bag 601, the user grasps handle 606 with their second hand and peels it from bag 601, exposing the sampling material.

While leaving their hand in the bag 601 the user can then wipe a surface with the sampling material using any number of techniques including linear passes followed by passes at 90 degrees and 45 degrees to the first pass as is outlined in the CDC sampling method, circular motion or other motions that will be known to and appreciated by those skilled in the art.

After sampling the user can grasp the inside bottom of bag 601 with their hand in the bag and using the second hand they can grasp the folded over bag material near the leak proof closure 602. By pulling with both hands towards each other—until passing each other—the user can then reinvert the bag 601.

Bag 601 can then be either transported to a laboratory or other location for extraction and analysis or extraction can be performed on site. For transport, bag 601 is sealed using leak proof closure 602 and placed inside of a secondary container prior to transport. For extraction of a dry sampling material, a liquid extraction buffer is added to the inside of the bag and the leak proof closure 602 is closed. For extraction of a wet sampling material, the leak proof closure 602 is simply closed prior to extraction.

To extract the sampling material, the user holds bag 601 horizontally with sampling material at the bottom of the bag. The bag 601 is then folded until the leak proof closure 602 end and the sampling port 603 end are touching or nearly touching. In this way sampling material is at the bottom of the now U-shaped bag 601. The user then grasps the sampling material in the palm of one hand, through the wall of bag 601, and tightly squeezes the sampling material—expelling most of the liquid sample out of the sampling material. The sampling material is then released so that the liquid flows back out of said sampling material. Continuing to hold bag 601 in the same configuration and orientation the user then repeats the squeeze and release method at least two more times.

The user then holds bag 601 vertically such that leak proof closure 602 is at the top of the bag 601 and the sampling port 603 is at the bottom of the bag 601. While holding bag 601 in this configuration and orientation the user then tightly squeezes the sampling material in the palm of one hand until nearly all of the liquid sample has been expelled. The user continues to hold bag 601 vertically until nearly all liquid sample has drained to sampling port 603. Again, continuing to hold bag 601 vertically the user now attaches a syringe or other sample extraction device to sampling port 603. The user then pulls back on the syringe barrel until nearly all of the liquid sampling has been withdrawn from bag 601.

Other methods of extraction, that are like the method described above or three repeated compress and release steps performed in the palm of the hand, can also be used with the large area surface sampler 600. These include the following: compressing and releasing the sampling material, through the wall of bag 601, between the palms of two hands; massaging the sampling material by circular and up and down compression and movement; and placing the bag 601 on a flat surface or in a bowl or other cupped surface and then compressing and releasing or massaging with one or two hands.

After removal of the liquid sample from bag 601, the user or laboratory may utilize any number of analysis methods that will be known to and appreciated by those skilled in the art including, but not limited to the following: culture/enumerate, polymerase chain reaction, quantitative real-time polymerase chain reaction, ATP-based methods, sequencing, antigen assays, biochemical assays, molecular detection methods, other rapid microbiological detection methods, mass spectrometry, chemical, cytometry, flow cytometry, or other growth-based microbiology methods to detect biological particles. Additionally, chemical analysis techniques may be used on the extracted sample to detect and identify particles of biological or non-biological nature.

FIG. 76 show an exemplary embodiment of a large area surface sampler 700 following removal of the protective cup. The large area surface sampler 700 is the sample as that shown in FIG. 5 and FIG. 6 but is shown after removal of the protective cup by the user. The bag 701 has sampling material 704 bonded to the inner bag 701 surface with leak proof closure 702 and opening 706. The leak proof closure 702 can be selected from a range of options that will be known to and appreciated by those skilled in the art, including but not limited to: zipper-type closures, folded waterproof bag type seals, adhesive seals, and twisted seals. The sampling material 704 is visible in this drawing because the protective cup has been removed from the sealing material 705. The sampling material 704 can be selected from a range of materials that will be known to and appreciated by those skilled in the art, including but not limited to: foam sponge materials; filter materials including, but not limited to: non-woven, woven, membrane, depth, electret, hydrophobic, hydrophilic filter materials. The sampling material 704 may be packaged in dry or wet state during manufacturing. The wetting agent can be selected from a range of liquids that will be known to and appreciated by those skilled in the art, including but not limited to: water, surfactant solutions, buffers, and solvents along with other additives such as neutralizing agents.

The sampling material 704 can be bonded to the bag using any number of techniques that will be appreciated by those skilled in the art, including but not limited to: tapes and adhesive films, contact adhesives, spray adhesives, thermal adhesives, pressure-sensitive adhesives, vibration welding, ultrasonic welding, and thermal welding. The sampling bag 701 can include a sampling port 703 which is attached to bag 701. The sampling port 703 can be selected from any number of devices that will be known to and appreciated by those skilled in the art, including but not limited to: self-opening valves, luer-lock valves, self-opening luer lock valves, self-sealing ports, septums, split septums, self-healing ports, spike ports, push-pull connectors, John Guest connectors, and push-to-connect.

The sampling material 704 can be left uncovered if desired or may be covered by a protective cup. If the sampling material 704 is covered, then it may be first filled with a wetting agent prior to sealing the protective cup against the bag 701 with sealing material 705. The protective cup may be of a thermoplastic or other materials that will be known to and appreciated by those skilled in the art. The protective cup can be sealed to the bag 701 using sealing material 705 between the cup sealing ring and the bag 701. The sealing ring 705 provides a non-permanent, removable seal, which can be selected from any number of options that will be known to those skilled in the art, including but not limited to: tapes and adhesive films, contact adhesives, spray adhesives, thermal adhesives, pressure-sensitive adhesives, vibration welding, ultrasonic welding, and thermal welding. Protective cup includes handle which is used to remove the protective cup from bag 701 prior to sampling.

Bag 701 is provided to users in the inverted state. In this configuration the sampling material 704 is on the outside of the inverted bag 701. The top of the bag 701 and leak proof closure 702 are left folded over as shown so that the user can grasp this material when reinverting the bag after sampling and before extraction. The bottom of the bag 701, near sampling port 703, can be left sticking into the inside of the bag 701, for another point for the user to grab when reinverting the bag 701.

To use the large area surface sampler 700, the user places their hand into bag 701 through the opening 706 at leak proof closure 702. The user may use the large area surface sampler 700 with the palm of the hand facing toward or away from sampling material 704. While keeping one hand in the bag 701, the user grasps the cup handle with their second hand and peels it from bag 701, exposing the sampling material 704.

While leaving their hand in the bag 701 the user can then wipe a surface with the sampling material 704 using any number of methods including linear passes followed by passes at 90 degrees and 45 degrees to the first pass as is outlined in the CDC sampling method, circular motion or other motions that will be known to and appreciated by those skilled in the art.

After sampling the user can grasp the inside bottom of bag 701 with their hand in the bag and using the second hand they can grasp the folded over bag material near the leak proof closure 702. By pulling with both hands towards each other—until passing each other—the user can then reinvert the bag 701.

Bag 701 can then be either transported to a laboratory or other location for extraction and analysis or extraction can be performed on site. For transport, bag 701 is sealed using leak proof closure 702 and placed inside of a secondary container prior to transport. For extraction of a dry sampling material 704, a liquid extraction buffer is added to the inside of the bag and the leak proof closure 702 is closed. For extraction of a wet sampling material 704, the leak proof closure 702 is simply closed prior to extraction.

To extract the sampling material 704, the user holds bag 701 horizontally with sampling material 704 at the bottom of the bag. The bag 701 is then folded until the leak proof closure 702 end and the sampling port 703 end are touching or nearly touching. In this way sampling material 704 is at the bottom of the now U-shaped bag 701. The user then grasps the sampling material 704 in the palm of one hand, through the wall of bag 701, and tightly squeezes the sampling material 704—expelling most of the liquid sample out of the sampling material 704. The sampling material 704 is then released so that the liquid flows back out of said sampling material 704. Continuing to hold bag 701 in the same configuration and orientation the user then repeats the squeeze and release method at least two more times.

The user then holds bag 701 vertically such that leak proof closure 702 is at the top of the bag 701 and the sampling port 703 is at the bottom of the bag 701. While holding bag 701 in this configuration and orientation the user then tightly squeezes the sampling material 704 in the palm of one hand until nearly all of the liquid sample has been expelled. The user continues to hold bag 701 vertically until nearly all liquid sample has drained to sampling port 703. Again, continuing to hold bag 701 vertically the user now attaches a syringe or other sample extraction device to sampling port 703. The user then pulls back on the syringe barrel until nearly all of the liquid sampling has been withdrawn from bag 701.

Other methods of extraction, that are like the method described above or three repeated compress and release steps performed in the palm of the hand, can also be used with the large area surface sampler 700. These include the following: compressing and releasing the sampling material 704, through the wall of bag 701, between the palms of two hands; massaging the sampling material 704 by circular and up and down compression and movement; and placing the bag 701 on a flat surface or in a bowl or other cupped surface and then compressing and releasing or massaging with one or two hands.

After removal of the liquid sample from bag 701, the user or laboratory may utilize any number of analysis methods that will be known to and appreciated by those skilled in the art including, but not limited to the following: culture/enumerate, polymerase chain reaction, quantitative real-time polymerase chain reaction, ATP-based methods, sequencing, antigen assays, biochemical assays, molecular detection methods, other rapid microbiological detection methods, mass spectrometry, chemical, cytometry, flow cytometry, or other growth-based microbiology methods to detect biological particles. Additionally, chemical analysis techniques may be used on the extracted sample to detect and identify particles of biological or non-biological nature.

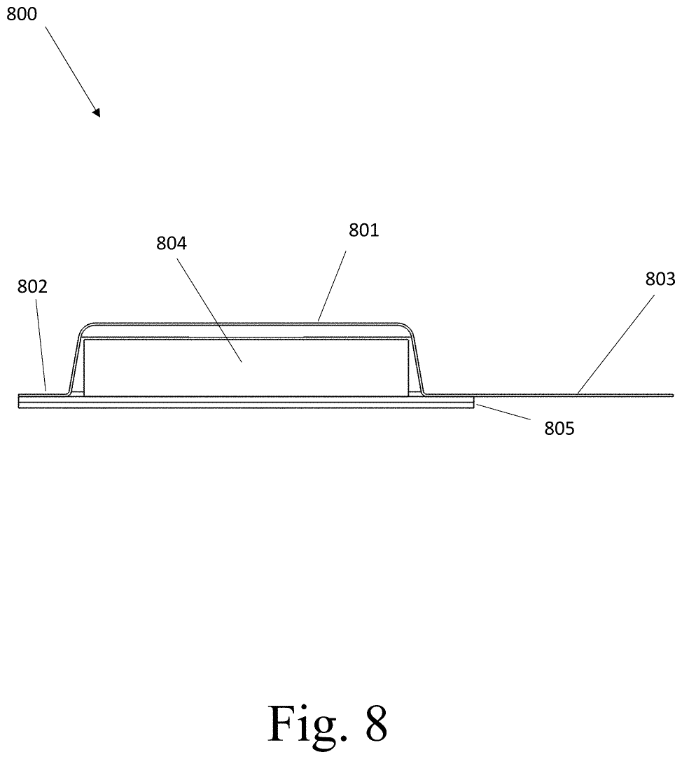

FIG. 8 shows an exemplary embodiment of sampling package component 800 of a large area surface sampler in the manufactured state. In the manufactured state—prior to use—the sampling package component 800 includes a sampling material 804 and protective cup 801 with handle 803 and flat sealing ring 802, which are all bonded to a sampling bag using one or more layers of sealing material 805.

The sampling material 804 can be selected from a range of materials that will be known to and appreciated by those skilled in the art, including but not limited to: foam sponge materials; filter materials including, but not limited to: non-woven, woven, membrane, depth, electret, hydrophobic, hydrophilic filter materials. The sampling material may be packaged in dry or wet state during manufacturing. The wetting agent can be selected from a range of liquids that will be known to and appreciated by those skilled in the art, including but not limited to: water, surfactant solutions, buffers, and solvents along with other additives such as neutralizing agents.

The sampling material 804 can be bonded to the bag using any number of methods or sealing materials 805 that will be known to and appreciated by those skilled in the art, including but not limited to: tapes and adhesive films, contact adhesives, spray adhesives, thermal adhesives, pressure-sensitive adhesives, vibration welding, ultrasonic welding, and thermal welding. The sealing material 805 is designed to provide a non-removable seal with the sampling material 804 that will withstand shipping and handling, air transport up to 20,000 feet, e-beam or gamma irradiation sterilization processes, and extended storage at room temperature and above.

The protective cup 801 may be of a thermoplastic or other materials that will be known to and appreciated by those skilled in the art. The protective cup 801 can be sealed to a sampling bag using sealing material 805 between the cup flat sealing ring 802 and the bag. In this case the sealing material 805 is a non-permanent, removable seal, which can be selected from any number of methods that will be known to those skilled in the art, including but not limited to: tapes and adhesive films, contact adhesives, spray adhesives, thermal adhesives, pressure-sensitive adhesives, vibration welding, ultrasonic welding, and thermal welding. Protective cup 804 includes handle 803 which is used to remove the protective cup 804 from bag 801 prior to sampling. The sealing material 805 is designed to provide a leak proof seal that will withstand shipping and handling, air transport up to 20,000 feet, e-beam or gamma irradiation sterilization processes, and extended storage at room temperature and above. The sealing material 805 must also allow straightforward release of the protective cup 801 from the bag when the user pulls on handle 803.

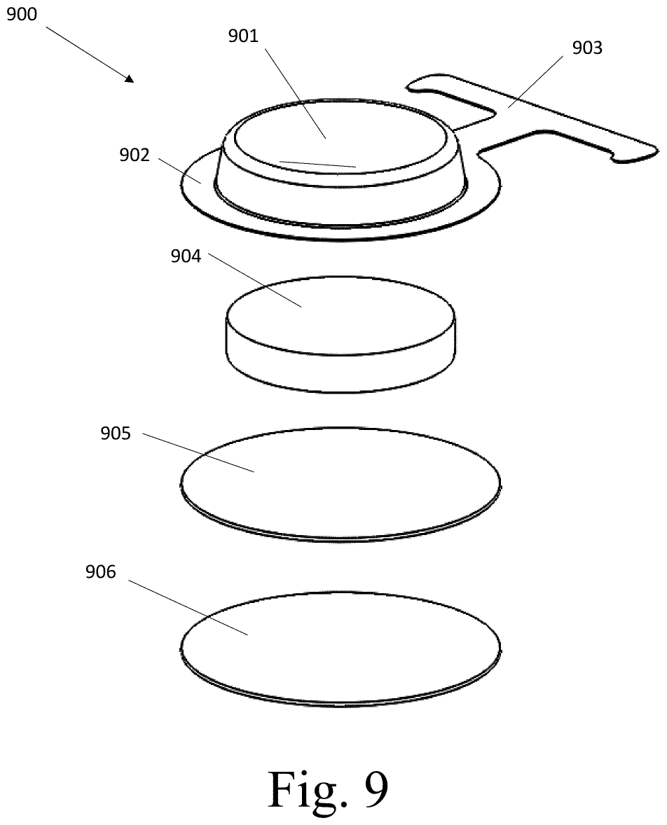

FIG. 9 shows an exploded view of an exemplary embodiment of sampling package component 900 of a large area surface sampler in the manufactured state. In the manufactured state—prior to use—the sampling package component 900 includes a sampling material 904 and protective cup 901 with handle 903 and flat sealing ring 902, which are all bonded to a sampling bag using one or more layers of sealing material 905 and 906.

The sampling material 904 can be selected from a range of materials that will be known to and appreciated by those skilled in the art, including but not limited to: foam sponge materials; filter materials including, but not limited to: non-woven, woven, membrane, depth, electret, hydrophobic, hydrophilic filter materials. The sampling material may be packaged in dry or wet state during manufacturing. The wetting agent can be selected from a range of liquids that will be known to and appreciated by those skilled in the art, including but not limited to: water, surfactant solutions, buffers, and solvents along with other additives such as neutralizing agents.

The sampling material 904 can be bonded to the bag using any number of methods or sealing materials 905 and 906 that will be known to and appreciated by those skilled in the art, including but not limited to: tapes and adhesive films, contact adhesives, spray adhesives, thermal adhesives, pressure-sensitive adhesives, vibration welding, ultrasonic welding, and thermal welding. The sealing material 905 and 906 is designed to provide a non-removable seal with the sampling material 904 that will withstand shipping and handling, air transport up to 20,000 feet, e-beam or gamma irradiation sterilization processes, and extended storage at room temperature and above.

The protective cup 901 may be of a thermoplastic or other materials that will be known to and appreciated by those skilled in the art. The protective cup 901 can be sealed to a sampling bag using sealing material 905 between the cup flat sealing ring 902 and the bag. In this case the sealing material 905 and 906 provides a non-permanent, removable seal, which can be selected from any number of methods that will be known to those skilled in the art, including but not limited to: tapes and adhesive films, contact adhesives, spray adhesives, thermal adhesives, pressure-sensitive adhesives, vibration welding, ultrasonic welding, and thermal welding. Protective cup 904 includes handle 903 which is used to remove the protective cup 904 from bag 901 prior to sampling. The sealing material 905 and 906 is designed to provide a leak proof seal that will withstand shipping and handling, air transport up to 20,000 feet, e-beam or gamma irradiation sterilization processes, and extended storage at room temperature and above. The sealing materials 905 and 906 must also allow straightforward release of the protective cup 901 from the bag when the user pulls on handle 903.

The foregoing disclosure of the exemplary embodiments of the present subject disclosure has been presented for purposes of illustration and description. It is not intended to be exhaustive or to limit the subject disclosure to the precise forms disclosed. Many variations and modifications of the embodiments described herein will be apparent to one of ordinary skill in the art in light of the above disclosure. The scope of the subject disclosure is to be defined only by the claims appended hereto, and by their equivalents.

Further, in describing representative embodiments of the present subject disclosure, the specification may have presented the method and/or process of the present subject disclosure as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process of the present subject disclosure should not be limited to the performance of their steps in the order written, and one skilled in the art can readily appreciate that the sequences may be varied and still remain within the spirit and scope of the present subject disclosure.

What is claimed is:

1. A surface sampling device, comprising;
a bag having an interior surface and an exterior surface, with a sealable opening at one end;
a collection material bonded to the bag;
a sampling port in the bag opposite the sealable opening;
wherein the bag is adapted for use in an inverted position with the interior surface on the outside;
wherein the bag is sized such that the user can place a hand into the bag, for use as a glove during sampling;
wherein the bag can be re-inverted and sealed after sampling to enable transport or extraction of the collected sample; and
wherein an extracted sample can be withdrawn from the bag through the sampling port.

2. The device in claim 1, wherein the sealable opening is a zip style closure, folded closure, twisted closure, or adhesive closure.

3. The device in claim 1, wherein the collection material includes one or more layers of hydrophobic foam, hydrophilic foam, open cell foam, non-woven, woven, membrane, fiber, depth, electret, hydrophobic, hydrophilic, or cover-web filtering materials.

4. The device in claim 3, wherein the hydrophobic foam is polyester, polyurethane or a mix of polyester and polyurethane.

5. The device in claim 1, wherein the collection material is bonded to the bag using one or more of adhesive, glue, tape, solvent bonding, ultrasonic weld, friction weld, laser welding, thermal bonding, or heat laminating.

6. The device in claim 1, wherein the collection material is bonded to the interior surface of the bag.

7. The device in claim 1, wherein the collection material is bonded to the exterior surface of the bag.

8. The device in claim 1, wherein the collection material is covered by a protective cup that is sealed to an external surface of the bag.

9. The device in claim 8, wherein the protective cup is removably bonded to the bag using one or more of adhesive, glue, tape, solvent bonding, ultrasonic weld, friction weld, laser welding, thermal bonding, or heat laminating.

10. The device in claim 8, wherein the collection material is filled with collection and extraction fluid before sealing the protective cup to the bag external surface.

11. The device in claim 10, wherein the collection and extraction fluid contains a buffer.

12. The device in claim 10, wherein the collection and extraction fluid contains a surfactant.

13. The device in claim 1, wherein the sampling port is bonded into the bag using one or more of adhesive, glue, tape, solvent bonding, ultrasonic weld, friction weld, laser welding, thermal bonding, or heat laminating.

14. The device in claim 1, wherein multiple bags are nested inside of each other such that a user can remove one bag at a time by placing a hand into the bag and removing it from said nest.

15. The device in claim 1, where in the collection material is used dry and a liquid extraction fluid is added to the bag after collecting a sample.

16. A surface sampling device, comprising;
a bag having an interior surface and an exterior surface, with a sealable opening at one end;
a collection material bonded to an exterior surface of the bag;
a protective cup sealed to the external surface of the bag and covering the collection material, the protective cup having a handle;
a sampling port in the bag opposite the sealable opening;
wherein the bag is adapted for use in an inverted position with the interior surface on the outside;
wherein the bag is sized such that the user can place a hand into the bag, for use as a glove during sampling;
wherein the bag can be re-inverted and sealed after sampling to enable transport or extraction of the collected sample; and
wherein an extracted sample can be withdrawn from the bag through the sampling port.

17. A method for collecting and detecting materials residing on surfaces, comprising:
providing a bag with a sealable opening with a wetted collection material bonded to an exterior surface, with a removable protective covering and a sampling port opposite the sealable opening, wherein the bag is provided to the user in an inverted position with the inside surface on the outside;
placing a hand into the bag and removing the protective covering;
sampling a surface area with the wetted collection material using an overlapping wiping method;
re-inverting the bag and sealing it closed with the sealable opening;
compressing and releasing the collection material one or more times to push fluid out of and back into the collection material;
attaching a syringe to the sample port;
holding the bag vertical with the sample port and syringe at the bottom and compressing the collection material to extract fluid therefrom;
withdrawing the liquid sample using the syringe; and
detecting the captured material.

18. The method of claim 17, wherein compressing and releasing the collection material takes place while holding the bag in a U-shaped configuration so that liquid pools around the collection material.

19. The method in claim 17, wherein a self-opening luer lock valve on the sample port closes when the syringe is removed.

20. The method of claim 17, further comprising using one or more of polymerase chain reaction, sequencing, antigen assays, biochemical assays, molecular detection methods, rapid microbiological detection methods, mass spectrometry, chemical, cytometry, flow cytometry, or growth-based microbiology methods to detect the materials residing on surfaces.
